(19) United States
(12) Patent Application Publication
(10) Pub. No.: US 2026/0094680 A1
(43) Pub. Date: Apr. 2, 2026

(54) DIGITAL MONITORING OF PATHOGEN LOAD IN LIVESTOCK

(71) Applicant: Evonik Operations GmbH, Essen (DE)

(72) Inventors: Emeka Ignatius IGWE, München (DE); Franziska LEVY, Mülheim (DE); Marco FECHER, Niedernberg (DE); Johann FICKLER, Mömlingen (DE); Frank THIEMANN, Nottuln (DE); Patrick BINGEMANN, Frankenthal (DE)

(73) Assignee: Evonik Operations GmbH, Essen (DE)

(21) Appl. No.: 18/870,565

(22) PCT Filed: Jun. 13, 2023
(86) PCT No.: PCT/EP2023/065782

§ 371 Date: Nov. 29, 2024

(30) Foreign Application Priority Data
Jun. 15, 2022 (EP) 22179243.5

(51) Int. Cl. G16H 10/60; A01K 29/00; G16H 15/00; G16H 50/30

(52) U.S. Cl. CPC G16H 10/60; A01K 29/005; G16H 15/00; G16H 50/30

(57) ABSTRACT

A method for monitoring pathogen load in livestock. The method uses a risk matrix to evaluate the risk of a pathogen load in livestock to cause a disease. A computer program product comprising code portions adapted for performing the method and a system for monitoring pathogen load in livestock are also described.

The present invention relates to a computer-implemented method for monitoring pathogen load in livestock, using a risk matrix for evaluating the risk of a pathogen load in livestock to cause a disease, a computer program product comprising code portions adapted for performing the method according to the present invention, and a system for monitoring pathogen load in livestock.

Infectious diseases of farm animals are one of the major threats to agriculture and can cause considerable damage at local, regional, and even at the international level both in industrialized and in developing countries. In the last two centuries considerable efforts have been invested in understanding the causes and pathogenesis of viral and bacterial diseases of domestic animals. These studies have introduced new methodologies for the diagnosis, treatment, and control of veterinary diseases. Importantly, research on veterinary pathogens also had a major impact in understanding basic biological processes of viruses and bacteria. In some cases, studies on veterinary pathogens have revolutionized biology and established entirely new disciplines.

The production cycle of livestock is often relatively short, in particular the production cycle of poultry. It therefore gives the farmers only a narrow time frame of opportunity for an effective intervention, especially when the risk of infectious diseases is eminent. Thus, infectious diseases can have a severe effect on the production cycle of livestock. Hence, in the first place, it needs a reliable and fast procedure for the detection of diseases-causing pathogens in livestock production, which gives the farmer a sufficient time frame of opportunity for an effective intervention.

However, the current best practice for the detection and/or monitoring of diseases-causing pathogens in livestock production involves several stand-alone processes with little or no connectivity and a time to result far exceeding the window of intervention. This practice limits the effective control of the entire process and poses some bottleneck to process standardization. Other methods like the use of sensors, cameras, and sound tracing devices assisted by artificial intelligence have also been reported to be viable approaches for monitoring livestock production processes. However, these approaches are in principle limited to the detection, estimation, and the tracking of the physical parameters, which may not be correlated with pathogen loads and/or disease occurrences of livestock farms.

Accordingly, there was still a need for a way of monitoring pathogen load in livestock, which gives the farmer a sufficient time frame of opportunity for an effective intervention.

It was found that this problem is solved by a method for monitoring pathogen load in livestock to find out the probability of a pathogen to cause a disease. In this method data being indicative for the identity and load of one or more pathogens in a livestock are evaluated by means of the risk matrix, which comprises, for each pathogen, a value for the limit of detection of the pathogen, a value for the lower limit for the occurrence of a disease caused by the pathogen, and a value for the threshold for the occurrence of a disease caused by the pathogen are comprised in a risk matrix, which allows for monitoring pathogen load in livestock. The risk matrix thus allows an assessment of the risk of the occurrence of a disease caused by a specific pathogen.

The risk matrix for evaluating the risk of a pathogen load in livestock to cause a disease,
comprises, for each pathogen, a value v_{D} for the limit of detection of the pathogen, a value v_{L} for the lower limit for the occurrence of a disease caused by the pathogen, and a value v_{T} for the threshold for the occurrence of a disease caused by the pathogen,
wherein
   v_{L} is n×v_{D} with n being a real number larger than 1, wherein the value for n is given by the likelihood of the pathogen in question to cause an infectious disease,
and wherein
   a value for the pathogen load of not more than v_{D} indicates that the risk for an infectious disease caused by the pathogen in question is unlikely,
   a value for the pathogen load being larger than v_{D} and not more than v_{L} indicates that the risk for an infectious disease caused by the pathogen in question is rare,
   a value for the pathogen load being larger than v_{L} and not more than v_{T} indicates that the risk for an infectious disease caused by the pathogen in question is possible, and
   a value for the pathogen load being larger than v indicates that the risk for an infectious disease caused by the pathogen in question is certain.

One object of the present invention is therefore a computer-implemented method for monitoring pathogen load in livestock, comprising the steps of
a) receiving a request from an input/output device of a user to connect to an operating unit, wherein said request comprises the input of at least the user identity,
b) the user request of step a) initiates the operating unit to perform at least the following steps
c) receiving or retrieving data from the input/output device of step a), wherein said data comprises at least
   C1) data Dₚₐₜ being indicative for the identity and load of one or more pathogens in a livestock, wherein the identity and the load of said pathogen are comprised in the data Dₚₐₜ,
   C2) identification I_{farm} of the farm, and/or farmhouse of the livestock of C1), and
   C3) the time point T_{data} of generation of the data of C1),
c′) determining the identity and/or load of the one or more pathogens in the data Dₚₐₜ, in case the identity and/or load of the pathogen is not already comprised in the data Dₚₐₜ,
d) evaluating the data of C1) and/or, if applicable, of c′), comprising the steps of
   d1) pulling a risk matrix from a database,
      wherein the risk matrix comprises, for each pathogen, a value v_{D} for the limit of detection of the pathogen, a value v_{L} for the lower limit of the occurrence of a disease caused by the pathogen, and a value v_{T} for the threshold of the occurrence of a disease caused by the pathogen, wherein
         v_{L} is n×v_{D} with n being a real number larger than 1, wherein the value for n is given by the likelihood of the pathogen in question to cause an infectious disease.
      and wherein
         a value for the pathogen load of not more than v_{D} indicates that the risk for an infectious disease caused by the pathogen in question is unlikely,
         a value for the pathogen load being larger than v_{L} and not more than v_{L} indicates that the risk for an infectious disease caused by the pathogen in question is rare,
         a value for the pathogen load being larger than v_{L} and not more than v_{T} indicates that the risk for an infectious disease caused by the pathogen in question is possible, and
         a value for the pathogen load being larger than v_{T} indicates that the risk for an infectious disease caused by the pathogen in question is certain
   d2) for every pathogen in the data Dₚₐₜ and/or whose identity is determined in step c′), reading off the values v_{D}, v_{L} and v_{T} from the risk matrix of step d1), and
   d3) for every pathogen, comparing the data Dₚₐₜ indicative for the pathogen load or the pathogen load determined in step c′) with the values v_{D}, v_{L} and v_{T} of step d2) to find out the probability of any of the pathogens of C1) or identified in c′) to cause a disease.

The value v_{D} for the limit of detection depends on the selectivity of the kit and/or the method used for the determination of the identity and the load of the pathogen in question. In addition, the individual conditions in farms or farmhouses may have an effect of varying strength on the sensitivity of the kit and/or the method used for the determination of the identity and the load of the pathogen in question. This also has an effect on the limit of detection and its value v_{D}. Therefore, the user of the risk matrix and of the method according to the present invention may choose n adequately to allow adequate and reliable conclusions for the values v_{L} and v_{T}. Accordingly, n is not subject to any limitations regarding its specific value. In any case n is a real number larger than 1, preferably n is a real number from larger than 1 to 10. It is preferred that the value of n is given by the likelihood of the pathogen in question to cause an infectious disease. Some pathogens have a larger likelihood to cause an infectious disease than other pathogens. Accordingly, the value of n is lower for those pathogens having a larger likelihood to cause an infectious disease, while the value of n is higher for those pathogens having a larger likelihood to cause an infectious disease.

The value v_{L} for the lower limit for the occurrence of a disease caused by the pathogen indicates the lower limit of the pathogen load for which it is typically not observed that a pathogen caused disease occurs.

The value v_{T} for the threshold for the occurrence of a disease caused by the pathogen indicates the threshold of the pathogen load for which it is typically observed that a pathogen caused disease occurs.

The values v_{L} and v_{T} depend on the individual pathogen in question. Some pathogens may cause diseases in livestock already at relatively low pathogen loads, while other pathogens may cause disease in livestock only at relatively high pathogen loads.

The request of step a) may further comprise the input of the identification I_{farm} of the farm, and/or farmhouse of the livestock.

In the context of the present invention the term livestock is used as known to the person skilled in the art and denotes the domesticated animals raised in an agricultural setting to provide labor and produce commodities such as meat, eggs, milk, fur, leather, and wool. Hence, in the context of the present invention the term livestock, both with respect to the risk matrix and the method according to the present invention, is not subject to any limitation regarding a specific type of animal. Rather, said term comprises all domesticated animals raised in an agricultural setting to provide labor and produce commodities such as meat, eggs, milk, fur, leather, and wool, e.g., cattle, sheep, goats, pigs, equine, and poultry. Preferred poultry is chicken and turkeys.

In an embodiment of the present invention the livestock is one or more of cattle, sheep, goats, pigs, equine, and poultry.

The method according to the present invention is neither limited to any specific domesticated animals or species nor to any specific pathogens or pathogenic load. Rather, the risk matrix used in the method according to the present invention may comprise a value v_{D} for the limit of detection of the pathogen, a value v_{L} for the lower limit of the occurrence of a disease caused by the pathogen, and a value v_{T} for the threshold of the occurrence of a disease caused by the pathogen for each pathogen causing a disease in livestock, e.g., cattle, sheep, goats, pigs, equine, and poultry. Alternatively, the risk matrix may comprise a value v_{D} for the limit of detection of the pathogen, a value v_{L} for the lower limit of the occurrence of a disease caused by the pathogen, and a value v_{T} for the threshold of the occurrence of a disease caused by the pathogen for each pathogen causing a disease in a specific livestock, i.e., in one of cattle, sheep, goats, pigs, equine, and poultry. In step d1) either a risk matrix for all domesticated animals or a risk matrix for the specific livestock in question is drawn from a database.

In the context of the present invention the term pathogen in the data Dₚₐₜ is used to denote that the identity of the pathogen and the load of said pathogen are comprised in the data Dₚₐₜ.

Alternatively, in case the identity of the pathogen is not comprised in the data Dₚₐₜ, the method further comprises the step c′) determining the identity and/or load of the one or more pathogens in the data Dₚₐₜ.

In the context of the present invention the term pathogen is used as known to the person skilled in the art and denotes any organism that can cause a disease. The term pathogen may also be referred to as an infectious agent, or simply a germ. Roughly speaking, pathogenic organisms are of five main types: viruses, bacteria, fungi, protozoa, and worms. The pathogen may cause a disease in the livestock in the first place and/or in humans who consume any animal-derived food products, contaminated with disease causing pathogens.

The five main types of pathogens and examples for them are further disclosed:

Bacteria are ubiquitous in nature; many species perform functions essential or beneficial to animal or human life, while others cause disease. Bacteria are unicellular organisms that lack an organized nucleus and contain no chlorophyll. They can have various shapes: spherical (coccus), rod-shaped (bacillus), comma-shaped (vibrio), spiral (spirillum), or corkscrew-shaped (spirochete), and may range from 0.5 to 5.0 μm in size. Some bacteria live in soil, plants, or water; others are parasites of humans, animals, and plants. Generally, bacteria can be classified into three groups based on their need for oxygen. Aerobic bacteria thrive in the presence of oxygen and require oxygen for continued growth and existence. Anaerobic bacteria thrive in oxygen-free environments. Facultative anaerobes can survive in either environment, although they prefer the presence of oxygen.

Important pathogenic bacteria include:

Campylobacter (meaning “curved bacteria”) is a genus of Gram-negative bacteria. Some Campylobacter species can infect humans, sometimes causing campylobacteriosis, a diarrheal disease in humans. The most known source for Campylobacter is poultry, but due to their diverse natural reservoir, Campylobacter spp. can also be transmitted via water. Other known sources of Campylobacter infections include food products, such as unpasteurized milk and contaminated fresh products. Sometimes the source of infection can be direct contact with infected animals, which often carry Campylobacter asymptomatically. At least a dozen species of Campylobacter have been implicated in human disease, with C. jejuni (80-90%) and C. coli (5-10%) being the most common. C. jejuni is recognized as one of the main causes of bacterial foodborne disease in many developed countries. It is the number one cause of bacterial gastroenteritis in Europe, with over 246,000 cases confirmed annually. C. fetus can cause spontaneous abortions in cattle and sheep and is an opportunistic pathogen in humans.

Salmonella is a genus of rod-shaped (bacillus) Gram-negative bacteria of the family Enterobacteriaceae. It causes salmonellosis, which is typically a food-borne disease. In humans, the most common symptoms are diarrhea, fever, abdominal cramps, and vomiting. Symptoms typically occur between 12 hours and 36 hours after exposure, and last from two to seven days. Occasionally more significant disease can result in dehydration. The old, young, and others with a weakened immune system are more likely to develop severe disease. Specific types of Salmonella can result in typhoid fever or paratyphoid fever. Salmonellosis is one of the most common causes of diarrhea globally. In 2015, 90,300 deaths occurred from nontyphoidal salmonellosis, and 178,000 deaths from typhoidal salmonellosis. In the United States, about 1.35 million cases and 450 deaths occur from non-typhoidal salmonellosis a year. In Europe, it is the second most common foodborne disease after campylobacteriosis.

Clostridium perfringens (formerly known as C. welchii, or Bacillus welchii) is a Gram-positive, rod-shaped, anaerobic, spore-forming pathogenic bacterium of the genus Clostridium. C. perfringens is ever-present in nature and can be found as a normal component of decaying vegetation, marine sediment, the intestinal tract of humans and other vertebrates, insects, and soil. Clostridium perfringens is one of the most common causes of food poisoning in the United States, alongside norovirus, Salmonella, Campylobacter, and Staphylococcus aureus. Infections due to C. perfringens show evidence of tissue necrosis, bacteremia, emphysematous cholecystitis, and gas gangrene, also known as clostridial myonecrosis.

Escherichia coli is a widely used gram-negative rod bacterium. Several non-pathogenic strains are part of the normal intestinal flora of humans. However, there are some serologically distinguishable strains that cause intestinal diseases in humans. In addition to the Enterohemorrhagic Escherichia coli (EHEC), which were first described in 1977, there are other pathogenic E. coli: enteropathogenic E. coli (EPEC), enterotoxin-forming E. coli (ETEC) and enteroinvasive E. coli (EIEC), enteroaggregative E. coli (EAEC) and diffusely adherent E. coli (DAEC).

Enterohemorrhagic Escherichia coli (EHEC) are certain disease-causing strains of the intestinal bacterium Escherichia coli (E. coli). The name prefix enterohemorrhagic (entero from ancient Greek \acute{ε}vτερov enteron—“intestine” and hemorrhagic for bleeding) indicates that EHEC can cause bloody diarrheal diseases (enterohemorrhagic colitis) in humans. It is verotoxin-producing Escherichia coli (VTEC) and synonymous with Shiga toxin-producing Escherichia coli (STEC) which cause clinical pictures in the patient. The pathogen, and the infectious diseases it causes, occur worldwide. The main reservoir of the pathogen is formed by ruminants, especially cattle, but also sheep, goats, and deer, in whose intestines they occur regularly without causing them diseases. In various countries of the world, there have been major outbreaks of EHEC, mainly due to the serotype O157: H7: For example, in 1982, when many people in the USA fell ill after eating insufficiently heated hamburgers, in 1996 in Japan with about 9000 sick schoolchildren after eating radish sprouts, and in 2006 from California in 26 states of the USA. In Germany, a continuous increase in the number of reports was observed in 2003 since the introduction of the nationwide reporting obligation in 1998.

Enteropathogenic E. coli (EPEC) cause severe diarrhea in young children, which is rare in industrialized societies but often responsible for childhood deaths in underdeveloped countries. Using EPEC adhesion factor (EAF), EPEC attach to the epithelial cells of the small intestine and then inject toxins into the enterocytes using a type III secretion system. There are also so-called atypical EPEC. They show the serotypes commonly used in STEC as well as virulence and pathogenicity factors such as the eae gene. However, they probably lost the Stx prophage and the associated stx genes characteristic of STEC.

Enterotoxic E. coli (ETEC) are more common causative agents of traveler's diarrhea (“Montezuma's revenge”). The reason for this disease is a heat-labile enterotoxin of the A/B type (LT I and LT II), as well as a heat-stable enterotoxin (ST). This 73 kDa protein has two domains, one of which binds to a G-ganglioside of the target cell (binding domain). The other domain is the active component, which activates adenylate cyclase similar to cholera toxin (about 80% gene homology). The ST, which is about 15-20 amino acids long, activates guanylate cyclase. Activation of adenylate cyclase and guanylate cyclase ends in secretory diarrhea, in which a lot of water and electrolytes are lost. The bacterium receives the genetic information from a lysogenic phage by transduction.

Enteroinvasive E. coli (EIEC) penetrate the epithelial cells of the colon and multiply there. Within the cell, actin tail formation occurs, with which they penetrate into neighboring epithelial cells like Listeria and Shigella. Inflammation and ulceration occur with the secretion of blood, mucus, and white blood cells (granulocytes). In addition, EIEC can release enterotoxins, which lead to electrolyte and water loss. The clinical picture is similar to bacterial dysentery with fever and bloody-mucous diarrhea, whereby a weakened symptomatology is often accompanied by watery diarrhea.

Enteroaggregative E. coli (EAggEC or EAEC) have the ability of auto-aggregating. They attach themselves to the small intestine epithelium with specific fimbriae. Characteristic is the increased mucus production of mucosa cells, which delays excretion. There is a diarrhea of the secretory type due to enterotoxins (EAST). EAEC causes both acute and chronic recurrent diarrheal diseases that can last for weeks. In addition to watery mucous diarrhea, there may also be fever and vomiting or bloody stools. In immunosuppressed people, e.g., HIV patients, EAEC is the most common causative agent of bacterial enteritis.

Other bacteria, however, of generally secondary role only, include Yersinia, Shigella, Brucella, and Leptospirosis.

Protozoa (singular protozoon or protozoan, plural protozoa or protozoans) is an informal term for a group of single-celled eukaryotes, either free-living or parasitic, that feed on organic matter such as other microorganisms or organic tissues and debris. Historically, protozoans were regarded as “one-celled animals”, because they often possess animal-like behaviors, such as motility and predation, and lack a cell wall, as found in plants and many algae. A protozoan infection, also called protozoonosis, or protozoan disease, is the active or passive penetration of protozoa (animal single-celled organisms) into an organism, their reproduction there and the subsequent reaction of the organism in the form of a disease.

Important pathogenic protozoa include:

Entamoeba histolytica (Amoebozoa), whose source of transmission are water and food, causes amoebiasis.

Balantidium coli (Ciliate), whose source of transmission are water and food, causes balantidiasis.

Toxoplasma gondii (Apicomplexa), whose source of transmission is amongst others undercooked meat, causes toxoplasmosis.

Cryptosporidium spp. (Apicomplexa), whose source of transmission are the fecal contamination of food and water, causes cryptosporidiosis.

Cyclospora cayetanensis (Apicomplexa), whose source of transmission are the fecal contamination of food and water, causes cyclosporiasis.

Viruses are small infectious agents that viruses that infect all life forms, from animals and plants to microorganisms, including bacteria and archaea. Viruses are found in almost every ecosystem on earth and are the most numerous type of biological entity. When infected, a host cell is often forced to rapidly produce thousands of copies of the original virus. When not inside an infected cell or in the process of infecting a cell, viruses exist in the form of independent particles, or virions, consisting of (i) the genetic material, i.e., long molecules of DNA or RNA that encode the structure of the proteins by which the virus acts; (ii) a protein coat, the capsid, which surrounds and protects the genetic material; and in some cases (iii) an outside envelope of lipids. The shapes of these virus particles range from simple helical and icosahedral forms to more complex structures. Most virus species have virions too small to be seen with an optical microscope and are one-hundredth the size of most bacteria.

There is a large number of important pathogenic viruses for all domesticated animals or for some of them. Some of these viruses, diseases they cause, and the domesticated species affected by them, are listed in the table below:

TABLE 1
List of exemplary viruses, the diseases they cause, and the domesticated species being
affected (P. Murcia et al., “Viral Pathogens of Domestic Animals and Their Impact
on Biology, Medicine and Agriculture”, Encyclopedia of Microbiology 2009, 805-819).

| Domesticated | | |
|---|---|---|
| species affected | Disease name | Virus |
| Multiple | Aujeszky's disease | Pseudorabies virus |
| | Bluetongue | Bluetongue virus |
| | Foot-and-mouth disease | Foot-and-mouth disease virus (serotypes |
| | | A, O, C, SAT1, SAT2, SAT3, Asia1) |
| | Japanese encephalitis | Japanese encephalitis virus |
| | Rabies | Rabies virus |
| | Rift Valley fever | Rift Valley fever virus |
| | Rinderpest | Rinderpest virus |
| | Vesicular stomatitis | Vesicular stomatitis virus |
| | West Nile fever | West Nile fever virus |
| Cattle | Bovine viral diarrhea | Bovine viral diarrhea virus |
| | Enzootic bovine leukosis | Bovine leukemia virus |
| | Infectious bovine | Bovine herpesvirus 1 |
| | rhinotracheitis/infectious pustular | |
| | vulvovaginitis | |
| | Lumpky skin disease | Lumpky skin disease virus |
| Sheep and goats | Caprine arthritis and encephalitis | Caprine arthritis and encephalitis virus |
| | Peste des petits ruminants | Peste-des-petits-ruminants virus |
| | Sheeppox and goatpox | Sheeppox and goatpox viruses |
| Equine | African horse sickness | African horse sickness virus |
| | Equine encephalomyelitis | Eastern equine encephalomyelitis virus |
| | (eastern) | |
| | Equine encephalomyelitis | Western equine encephalomyelitis virus |
| | (western) | |
| | Equine infectious anemia | Equine infectious anemia virus |
| | Equine influenza | Equine influenza virus |
| | Equine rhinopneumonitis | Equine herpesvirus 4 |
| | Equine viral arteritis | Equine arteritis virus |
| | Venezuelan equine | Venezuelan equine encephalomyelitis |
| | encephalomyelitis | virus |
| Swine | African swine fever | African swine fever virus |
| | Classical swine fever | Classical swine fever virus |
| | Nipah virus encephalitis | Nipah virus |
| | Porcine reproductive and | Porcine reproductive and respiratory |
| | respiratory syndrome | syndrome virus |
| | Swine vesicular disease | Swine vesicular disease virus |
| | Transmissible gastroenteritis | Transmissible gastroenteritis virus of |
| | | swine |
| Avian (poultry) | Avian infectious bronchitis | Avian infectious bronchitis virus |
| | Avian infectious laryngotracheitis | Infectious laryngotracheitis virus |
| | Duck hepatitis | Duck hepatitis virus |
| | Avian influenza | High and low pathogenic avian influenza |
| | | viruses |
| | Infectious bursal disease | Infectious bursal disease virus |
| | (Gumboro disease) | |
| | Marek's disease | Marek's disease virus |
| | Newcastle disease | Newcastle disease virus |
| | Turkey rhinotracheitis | Avian metapneumovirus |

The method according to the present invention is in particular useful for poultry, because of their relatively short life cycle. When poultry is the livestock in the method according to the present invention, the pathogen is preferably one or more of Campylobacter, Salmonella, Clostridium perfringens, Escherichia coli, Enterohemorrhagic Escherichia coli, Enteropathogenic E. coli, Enterotoxic E. coli, Enteroinvasive E. coli, Enteroaggregative E. coli, avian infectious bronchitis virus, infectious laryngotracheitis virus, duck hepatitis virus, high and low pathogenic avian influenza viruses, infectious bursal disease virus, Marek's disease virus, Newcastle disease virus, and avian metapneumovirus.

In step c) of the method according to the present invention data Dₚₐₜ are received or retrieved from the input/output device of step a), which are indicative for the identity and load of one or more pathogens in a livestock. Such data are preferably the concentration of a pathogen per mass, and/or the number of DNA copies of a pathogen per mass.

In an embodiment of the method according to the present invention the data of C1) comprises the concentration of a pathogen per mass, and/or the number of DNA copies of a pathogen per mass.

It is preferred that the probability of any of the pathogens of C1) or identified in c′) to cause a disease found in step d3) is sent to the input/output device of step a).

Alternatively or additionally, it is also possible to continue working with said findings. It is beneficial to store the data Dₚₐₜ, the identification I_{farm} of the farm, and/or farmhouse, and the time point T_{data} of generation of the data Dₚₐₜ, received or retrieved in step c) together with the finding of step d) in a dataset Dᵤₛₑᵣ, for the user of step a). When the method of claim 1 is repeatedly carried out for the same user identity, it is beneficial to store the data Dₚₐₜ, the identification I_{farm} of the farm, and/or farm-house, and the latest time point T_{data} of generation of the data, received or retrieved in step c), together with the finding of step d3) in the same dataset Dᵤₛₑᵣ as in step c1). This allows to provide for an aggregation of relevant data, e.g., for the representation of the development of the collected Dₚₐₜ and the identification of the trends in the development of the pathogen load in the livestock in question.

In a further embodiment the method according to the present invention further comprises the steps
d4) storing the data Dₚₐₜ, the identification I_{farm} of the farm, and/or farmhouse, and the time point T_{data} of generation of the data Dₚₐₜ, received or retrieved in step c) together with the finding of step d3) in a dataset Dᵤₛₑᵣ, for the user of step a), and
d5) when the method according to the present invention is repeatedly carried out for the same user identity, storing the data Dₚₐₜ, the identification I_{farm} of the farm, and/or farmhouse, and the latest time point T_{data} of generation of the data, received or retrieved in step c), together with the finding of step d3) in the same dataset Dᵤₛₑᵣ as in step c1).

It is further beneficial that for each farm and/or farmhouse of the user of step a) the data Dₚₐₜ, findings and latest time point T_{data} are grouped as individual reports in a list, with the reports having the latest time points at the top of the list. This gives the user a complete overview over the situation in each of his farms and/or farmhouses at the time points of data generation.

In another embodiment the method according to the present invention further comprises the steps
e) the user request of step a) initiates the operating unit to perform at least the steps
   e1) for each farm and/or farmhouse of the user of step a), pulling the data Dₚₐₜ, the findings and the latest time point T_{data} of generation of the data stored in step d4) or d5) from the dataset Dᵤₛₑᵣ for said user,
   e2) for each farm and/or farmhouse, grouping the data Dₚₐₜ, findings and latest time point T_{data} pulled in step e1) as individual reports in a list, with the reports having the latest time points T_{data} at the top of the list, and
   e3) sending the list of reports obtained in step e2) to the input/output device of the user.

Preferably, the reports indicate the probability of a pathogen in question to cause a risk according to the risk matrix according to the present invention. It is further preferred that, for every pathogen in question, i.e., in the data Dₚₐₜ, the risk for an infectious disease caused by the pathogen in question is categorized as
unlikely, when the data Dₚₐₜ being indicative for the load of the pathogen is not more than v_{D} for said pathogen in the risk matrix,
rare, when the data Dₚₐₜ being indicative for the load of the pathogen is larger than v_{D} and not more than v_{L} for said pathogen in the risk matrix,
possible, when the data Dₚₐₜ being indicative for the load of the pathogen is larger than v_{L} and not more than v_{T} for said pathogen in the risk matrix, and
certain, when the data Dₚₐₜ being indicative for the load of the pathogen is larger than v_{T} for said pathogen in the risk matrix.

In general, each value, in particular any measured value, is subject to a measurement error or an inaccuracy in the method of determination. The method according to the present invention therefore has to consider any measurement error or inaccuracies in the method of determination, when assessments are made regarding the risk for an infectious disease without losing any validity in the assessments. This is in particular relevant when the pathogen load is very close to the values v_{L} and v_{T} and the risk for an infectious disease caused by the pathogen in question could be either categorized as possible or certain, which makes a big difference. It was found that this problem is solved by considering a p-value of 0.05 as statistically relevant difference from the values v_{L} and v_{T}. In detail, the risk for an infectious disease caused by the pathogen in question is categorized in the next higher risk class, when the p-value from the comparison of the load for the pathogen in question and the reference value for the lower risk class is <0.05. For example, the load for the pathogen in question is categorized as certain, when said load has a p-value of <0.05 compared to the value v_{T}.

It is preferred that the identity and the load of the one or more pathogen in question are given in specific colors indicating the degree of risk or in a specific pattern according to a code indicating the degree of risk. This has the advantage of a better awareness of any potential risk for the occurrence of a pathogen caused disease. For example,
when the risk for an infectious disease caused by the pathogen in question is unlikely, the identity of the pathogen, and/or the data Dₚₐₜ being indicative for its load are colored in green,
when the risk for an infectious disease caused by the pathogen in question is rare, the identity of the pathogen, and/or the data Dₚₐₜ being indicative for its load are colored in yellow,
when the risk for an infectious disease caused by the pathogen in question is possible, the identity of the pathogen, and/or the data Dₚₐₜ being indicative for its load are colored in orange, and
when the risk for an infectious disease caused by the pathogen in question is certain, the identity of the pathogen, and/or the data Dₚₐₜ being indicative for its load are colored in red.

In the next step, the user can select from the thus provided list of reports. For the report selected in step e4), the load of the livestock with every pathogen in the data Dₚₐₜ for each time point T_{data} of generation of the data Dₚₐₜ is plotted into a diagram and for every pathogen in data Dₚₐₜ, the values v_{L} and v_{T} of the occurrence of the disease caused by the pathogen is also plotted into the diagram. Since the data are plotted into the diagram for each time point of data generation, this procedure facilitates the identification of trends in the development of the pathogen load in livestock both in general, i.e., the overall pathogen load, and in particular, i.e., the specific load with a specific pathogen. Further, the overall risk of the occurrence can be identified when the value(s) v and/or v-read off from the risk matrix also is/are plotted into the diagram.

In a preferred embodiment step e) of the method according to the present invention further comprises the steps
e4) receiving a selection of a report from the input/output device of the user,
e5) for the report selected in step e4), plotting the load of the livestock with every pathogen in the data Dₚₐₜ for each time point T_{data} of generation of the data Dₚₐₜ into a diagram,
e6) pulling the risk matrix according to the present invention, preferably the risk matrix of step d1) of the method according to the present invention, from a database,
e7) for every pathogen in the data Dₚₐₜ, reading off the value(s) v_{L} and/or v_{T} for the one or more pathogens from the risk matrix according to the present invention, preferably the risk matrix of step d1) of the method according to the present invention, and
e8) plotting the value(s) v_{L} and/or v_{T} read off in step e7) into the diagram obtained in step e5).

Once the user has read the selected report, the user is typically no longer interested in reading the same old reports again. Rather, the user's main focus is on the most recent reports and data. In order to avoid that the user has to go through any old reports again, the method according to the present invention receives a read confirmation for the report selected in step e4) from the input/output device of the user and stores the read confirmation for the report together with the corresponding data. When at least the step e), or the method according to the present invention, is carried out repeatedly, the read confirmation is also pulled from the dataset Dᵤₛₑᵣ, and all reports without a read confirmation are ranked in a first list, with the reports having the latest time points at the top of said first list, and all other reports are ranked in a second list, with the reports having the latest time points at the top of said second list.

In a further preferred embodiment, the step e) of the method according to the present invention further comprises the steps
e9) if applicable, receiving a read confirmation for the report selected in step e4) from the input/output device of the user, and storing the read confirmation for the report together with the corresponding Dₚₐₜ, findings and latest time point T_{data} in the dataset Dᵤₛₑᵣ, and
e10) when at least the step e) is carried out repeatedly, the step e1) further comprises pulling a read confirmation from the dataset Dᵤₛₑᵣ, and the step e2) further comprises ranking all reports without a read confirmation in a first list, with the reports having the latest time points at the top of said first list, and all other reports in a second list, with the reports having the latest time points at the top of said second list.

It also beneficial for the user that the method according to the present invention give advice for the further samplings, specifically giving advice for the date for scheduled samplings, based on the dates of time point T_{data} of C3).

In yet another preferred embodiment of the method according to the present invention the report of step e2) further comprises the dates for scheduled samplings based on the dates of time point T_{data} of C3).

In still another preferred embodiment the method according to the present invention further comprises the step
f) planning a new cycle for monitoring the pathogen load in livestock, comprising the steps of
   f1) receiving a selection or the input of the farm and/or farmhouse for which a new cycle is to be planned,
   f2) receiving a selection or the input of the start date for the new cycle, and
   f3) receiving a section or the input of the livestock age at the time of starting the new cycle and the scope of the new cycle.

In order to avoid any overlaps of new cycles for monitoring the pathogen load in livestock with the cycles for monitoring the pathogen load in livestock which are active, it is beneficial to indicate these active cycles in step f2).

In a preferred embodiment the step f2) further comprises the indication of the cycles for monitoring the pathogen load in livestock which are active.

It may be also beneficial for the user to get information on the pathogen load for all farms and/or farmhouses of him, for example the mean pathogen status for all farms and/or farmhouses and the mean condition risk for all of his harms and/or farmhouses.

Preferably, the method according to the present invention therefore further comprises the step
g) receiving a request from the input/output device of a user to aggregate reports, comprising the steps of
   g1) for all farms and/or farmhouses of the user, pulling the load of the livestock with each pathogen in each farm and/or farmhouse from the data Dₚₐₜ from the dataset Dᵤₛₑᵣ, forming the mean of the load with each pathogen, to get the mean pathogen status for all farms and/or farmhouses.

To get the mean condition risk for all farms and/or farmhouses, the step g1) is preferably followed by the following steps
g2) pulling the risk matrix according to the present invention, in particle the risk matrix of step d1) of the method according to the present invention, from a database, and
g3) for all farms and/or farmhouses, reading off the values v_{L} and/or v_{T} for each pathogen in data Dₚₐₜ, and forming the mean of the values v_{L} and/or v_{T} to get the mean condition risk v_{L,mean} and/or v_{T,mean} for all farms and/or farmhouses.

The steps g1) to g3) can also be combined in one embodiment.

In another embodiment the method according to the present invention further comprises the step
g) receiving a request from the input/output device of a user to aggregate reports, comprising the steps of
   g1) for all farms and/or farmhouses of the user, pulling the load of the livestock with each pathogen in each farm and/or farmhouse from the data Dₚₐₜ from the dataset Dᵤₛₑᵣ, forming the mean of the load with each pathogen, to get the mean pathogen status for all farms and/or farmhouses,
   g2) pulling the risk matrix according to the present invention, preferably the risk matrix of step d1) of the method according to the present invention, from a database, and
   g3) for all farms and/or farmhouses, reading off the values v_{L} and/or v_{T} for each pathogen in data Dₚₐₜ, and forming the mean of the values v_{L} and/or v_{T} to get the mean condition risk v_{L,mean} and/or v_{T,mean} for all farms and/or farmhouses.

The method according to the present invention can be used on any conceivable computer device that allows to perform said method. The objects of the present invention are not subject to any limitation regarding the computer device, provided that it allows for receiving the request of step a), receiving or retrieving the data of step c), and comprising or having access to the database with the risk matrix of the method according to the present invention. For example, the computer device can be a stationary or mobile computer. Examples for a stationary computer are a desktop computer and a network computer. Examples for a portable computer are a laptop, a notebook, a tablet, and a smartphone.

A further object of the present invention is therefore a computer program product comprising code portions adapted for performing the method according to the present invention, when said program is loaded into a computer device.

Preferably, the computer program product comprising code portions either comprises or has access to a database with the risk matrix according to the present invention.

Alternatively, the program for the method according to the present invention and/or code portions for performing said method can be stored on a computer usable medium, such as a USB stick, a memory card or a CD-ROM. In that case, the program and/or the code portions is/are stored as computer readable program means which causes a computer to perform the method according to the present invention.

Yet another object of the present invention is therefore a computer program product stored on a computer usable medium, comprising computer readable program means for causing a computer to perform the method according to the present invention.

Preferably, the computer program product stored on a computer usable medium either comprises or has access to a database with the risk matrix according to the present invention.

The method according to the present invention can be implemented to give a system for monitoring pathogen load in livestock.

An object of the present invention is therefore also a system for monitoring pathogen load in livestock, comprising an operating unit adapted to perform the method according to the present invention, and comprising or having access to the database with the risk matrix of the method according to the present invention, preferably the matrix of step d1) of the method according to the present invention.

1. A computer-implemented method for monitoring pathogen load in livestock, comprising:
a) receiving a request from an input/output device of a user to connect to an operating unit, wherein the request comprises the input of the user identity;
b) the user request of a) initiates the operating unit to perform the following;
c) receiving or retrieving data from the input/output device of a), wherein the data comprises;
C1) data Dₚₐₜ being indicative for the identity and load of one or more pathogens in a livestock, wherein the identity and the load of the pathogen are comprised in the data Dₚₐₜ;
C2) identification I_{farm} of a farm, and/or farmhouse of the livestock of C1); and
C3) the time point T_{data} of generation of the data of C1),
d) evaluating the data of C1), by a method comprising:
d1) pulling a risk matrix from a database, wherein the risk matrix comprises, for each pathogen, a value v_{D} for the limit of detection of the pathogen, a value v_{L} for the lower limit of the occurrence of a disease caused by the pathogen, and a value v_{T} for the threshold of the occurrence of a disease caused by the pathogen, and
wherein;
v_{L} is n×v_{D} with n being a real number larger than 1, wherein the value for n is given by the likelihood of the pathogen in question to cause an infectious disease
a value for the pathogen load of not more than v_{D} indicates that the risk for an infectious disease caused by the pathogen in question is unlikely,
a value for the pathogen load being larger than v_{D} and not more than vindicates that the risk for an infectious disease caused by the pathogen in question is rare,
a value for the pathogen load being larger than v_{D} and not more than v indicates that the risk for an infectious disease caused by the pathogen in question is possible, and
a value for the pathogen load being larger than v_{T} indicates that the risk for an infectious disease caused by the pathogen in question is certain;
d2) for every pathogen in the data Dₚₐₜ, reading off the values v_{D}, v_{L}, and v_{T} from the risk matrix of d1);
d3) for every pathogen, comparing the data Dₚₐₜ indicative for the pathogen load) with the values v_{D}, v_{L}, and v_{T} of d2) to find out the probability of any of the pathogens of C1) to cause a disease;
d4) storing the data Dₚₐₜ, the identification/farm of the farm, and/or farmhouse, and the time point T_{data} of generation of the data D_{Pat}, received or retrieved in c) together with the finding of d3) in a dataset Dᵤₛₑᵣ, for the user of a); and
d5) when the method is repeatedly carried out for the same user identity, storing the data Dₚₐₜ, the identification I_{farm} of the farm, and/or farmhouse, and the latest time point T_{data} of generation of the data, received or retrieved in c), together with the finding of d3) in the same dataset Dᵤₛₑᵣ as in d4);
e) the user request of a) initiates the operating unit to perform the following:
e1) for each farm and/or farmhouse of the user of a), pulling the data Dₚₐₜ, the findings and the latest time point T_{data} of generation of the data stored in d4) or d5) from the dataset Dᵤₛₑᵣ for the user;
e2) for each farm and/or farmhouse, grouping the data Dₚₐₜ, findings and latest time point T_{data} pulled in e1) as individual reports in a list, with the reports having the latest time points T_{data} at the top of the list;
e3) sending the list of reports obtained in e2) to the input/output device of the user,
e4) receiving a selection of a report from the input/output device of the user;
e5) for the report selected in e4), plotting the load of the livestock with every pathogen in the data Dₚₐₜ for each time point T_{data} of generation of the data Dₚₐₜ into a diagram;
e6) pulling the risk matrix of d1) from a database;
e7) for every pathogen in the data Dₚₐₜ, reading off the value(s) v_{L} and/or v_{T} for the one or more pathogens from the risk matrix of d1); and
e8) plotting the value(s) v_{L} and/or v_{T} read off in e7) into the diagram obtained in e5).

2. The method of claim 1, wherein the livestock is one or more of cattle, sheep, goats, pigs, equine, and poultry.

3. The method of claim 1, wherein the data of C1) comprises the concentration of a pathogen per mass, and/or the number of DNA copies of a pathogen per mass.

4-6. (canceled)

7. The method of claim 1, further comprising:
e9) if applicable, receiving a read confirmation for the report selected in e4) from the input/output device of the user, and storing the read confirmation for the report together with the corresponding Dₚₐₜ, findings and latest time point T_{data} in the dataset Dᵤₛₑᵣ, and
e10) when e) is carried out repeatedly, e1) further comprises pulling a read confirmation from the dataset Dᵤₛₑᵣ, and e2) further comprises ranking all reports without a read confirmation in a first list, with the reports having the latest time points at the top of the first list, and all other reports in a second list, with the reports having the latest time points at the top of the second list.

8. The method of claim 1, wherein the report of e2) further comprises the dates for scheduled samplings based on the dates of time point T_{data} of C3).

9. The method according to claim 1, further comprising:
f) planning a new cycle for monitoring the pathogen load in livestock, comprising:
f1) receiving a selection or the input of the farm and/or farmhouse for which a new cycle is to be planned;
f2) receiving a selection or the input of the start date for the new cycle; and
f3) receiving a section or the input of the livestock age at the time of starting the new cycle and the scope of the new cycle.

10. The method of claim 9, wherein the f2) further comprises the indication of the cycles for monitoring the pathogen load in livestock which are active.

11. The method of claim 1, further comprising:
g) receiving a request from the input/output device of a user to aggregate reports, comprising:
g1) for all farms and/or farmhouses of the user, pulling the load of the livestock with each pathogen in each farm and/or farmhouse from the data Dₚₐₜ from the dataset Dᵤₛₑᵣ, forming the mean of the load with each pathogen, to get the mean pathogen status for all farms and/or farmhouses.

12. The method of claim 1, further comprising:
g) receiving a request from the input/output device of a user to aggregate reports, comprising:
g1) for all farms and/or farmhouses of the user, pulling the load of the livestock with each pathogen in each farm and/or farmhouse from the data Dₚₐₜ from the dataset Dᵤₛₑᵣ, forming the mean of the load with each pathogen, to get the mean pathogen status for all farms and/or farmhouses;
g2) pulling the risk matrix from a database; and
g3) for all farms and/or farmhouses, reading off the values v_{L} and/or v_{T} for each pathogen in data Dₚₐₜ, and forming the mean of the values v_{L} and/or v_{T} to get the mean condition risk v_{L,mean} and/or v_{T,mean} for all farms and/or farm-houses.

13. A computer program product comprising code portions adapted for performing the method of claim 1, when the program is loaded into a computer device.

14. A computer program product stored on a computer usable medium, comprising computer readable program means for causing a computer to perform the method of claim 1.

15. A system for monitoring pathogen load in livestock, comprising an operating unit adapted to perform the method according to claim 1, and comprising or having access to the database with the risk matrix.
